(19) United States
(12) Patent Application Publication
(10) Pub. No.: US 2004/0236280 A1
(43) Pub. Date: Nov. 25, 2004

(54) Tubular device for insertion into a hollow organ, and method to form same

(76) Inventors: Lisette M. Rice, Tucson, AZ (US); James Bardwell, Cincinnati, OH (US)

Correspondence Address: Dale F. Regelman, Law Office of Dale F. Regelman, P.C., 4231 S. Fremont Avenue, Tucson, AZ, 85714, US

(21) Appl. No.: 10/851,363

(22) Filed: May 21, 2004

Related U.S. Application Data
(60) Provisional application No. 60/473,046, filed on May 22, 2003.

(51) Int. Cl. A61M 29/00

(52) U.S. Cl. 604/104; 428/35.3; 264/632

(57) ABSTRACT

A device, and method for making same, for insertion into a hollow organ of an animal, including humans is disclosed. The device includes a first tubular member having a first diameter, a proximal portion, and a distal portion formed to include a first aperture. The device further includes a tubular access assembly comprising a proximal portion having a second diameter and formed to include a second aperture, and a distal portion having said the diameter, where that distal portion of the access assembly is continuously attached to the proximal portion of the first tubular member, and where the second diameter is greater than or equal to the first diameter. The device further includes an inflatable internal retention assembly disposed around the distal portion of the first tubular member, where that inflatable retention assembly comprises a fluorinated siloxane elastomer.

### FIELD OF THE INVENTION

The invention relates to a tubular device for insertion into a hollow organ of an animal, including humans. In certain embodiments, the invention comprises a gastric tube. In certain embodiments, the invention comprises a nasogastric tube. In certain embodiments, the invention comprises a jejunal tube.

### BACKGROUND OF THE INVENTION

Medical devices comprising one or more tubes are used for introducing fluids into one or more hollow organs of an animal, including a human, and/or for evacuating fluids therefrom. For example, gastrointestinal feeding systems are frequently used for long-term-tube-fed patients who are unable to take nutrition orally. Such gastrointestinal systems comprise a source of nutrition interconnected with a gastrostomy tube.

One end of the gastrostomy tube is normally inserted into the patient's stomach via a stoma formed in the patient's abdominal wall. That inserted end includes an internal retention assembly. The internal retention assembly holds the inserted gastronomy tube in place by capturing the organ wall and abdominal wall between the internal retention assembly and an external retention assembly, sometimes called a “wound barrier,” disposed around the gastronomy tube external to the patient's outer abdominal wall. A hollow tubular member comprising one or more lumens passes through the respective retention assemblies. The tubular member provides a fluid pathway between the feeding set connected to a source of nutrition and the gastrointestinal tract of the patient.

As those skilled in the art will appreciate, a gastrostomy tube is sometimes referred to a “G” tube. A nasogastric tube comprises a different device. A nasogastric tube is passed through the nostril, nasopharynx, oropharynx, and esophagus and into the stomach. As those skilled in the art will further appreciate, a nasogastric tube is sometimes referred to as an “NG” tube. A jejunal tube is designed for placement into the small bowel using endoscopic techniques. As those skilled in the art will appreciate, a jejunal tube is sometimes referred to as a “J” tube. The lengths of NG tubes, G tubes, and J tubes differ. These various devices may include differing input ports, differing input port designs, differing number of lumen, and the like. Nevertheless, each of these various devices generally include an internal retention assembly.

In prior art devices, such an internal retention assembly typically comprises an inflatable balloon-type assembly formed from silicone elastomer, such as for example polydimethylsiloxane. After the tube is inserted into a hollow organ, the internal retention assembly is inflated thereby fixturing the tube in the hollow organ. Thereafter, the inflated internal retention assembly is continuously exposed to various body fluids. For example, if a gastric tube is placed into a patient's stomach, the inflated internal retention assembly is continuously exposed to fluids having a pH as low as 0.9.

What is needed is a device for insertion into a hollow organ, whether a gastric tube, nasogastric tube, jejunal tube, or the like, which includes an inflatable internal retention assembly that has increased resistance to body fluids, and more particularly increased resistance to acidic fluids found in a patient's stomach. Applicants' invention meets this need by disposing an inflatable internal retention assembly comprising a fluorinated siloxane elastomer on various tubular assemblies formed for insertion into a hollow organ of an animal, including humans.

### SUMMARY OF THE INVENTION

Applicants' invention includes a device for insertion into a hollow organ of an animal, including humans. Applicants' device includes a tubular member having a first diameter, a proximal portion, and a distal portion formed to include a first aperture. Applicants' device further includes a tubular access assembly comprising a proximal portion having a second diameter and formed to include a second aperture, and a distal portion having the first diameter, where that distal portion of the access assembly is continuously attached to the proximal portion of the tubular member, and where the second diameter is greater than or equal to the first diameter.

Applicants' device further includes an inflatable internal retention assembly disposed around the distal portion of the tubular member, where that inflatable retention assembly comprises a fluorinated siloxane. The tubular access assembly in combination with the tubular member defines a lumen interconnecting the first aperture and the second aperture.

### BRIEF DESCRIPTION OF THE DRAWINGS

The invention will be better understood from a reading of the following detailed description taken in conjunction with the drawings in which like reference designators are used to designate like elements, and in which:

FIG. 1A is a cross sectional view of Applicants' gastric tube having a deflated internal retention assembly;

FIG. 1B is a cross-sectional view of one embodiment of the gastric tube of FIG. 1 which includes a second tubular member disposed inside a first tubular member;

FIG. 1C is a cross-sectional view of another embodiment of the gastric tube of FIG. 1 which includes a second tubular member disposed external to a first tubular member;

FIG. 2 is a cross sectional view of the gastric tube of FIG. 1, wherein the internal retention assembly is inflated;

FIG. 3 is a flow chart summarizing the steps of Applicants' method to form the gastric tube of FIG. 1.

### DETAILED DESCRIPTION OF THE PREFERRED EMBODIMENTS

The invention will be described as embodied in a gastric tube. The following description of Applicants' invention is not meant, however, to limit that invention to gastric tubes only, as the invention herein can be applied to gastronomy tubes, NG tubes, G tubes, J tubes, and the like.

One embodiment of Applicants' device comprises a gastric tube. Applicants' gastric tube includes an inflatable internal retention assembly. Such inflated assemblies are sometimes called “balloons.” Applicants' internal retention assembly comprises an inflatable, toroidal-shaped, fluorinated siloxane disposed around the distal end, i.e. the insertable end, of Applicants' device. When this internal retention assembly is deflated, the distal end of Applicants' device has a diameter about equal to that of the diameter of the tubular member itself. Thus when deflated, Applicants' device may be safely and conveniently inserted in, or removed from, a patient. After insertion into, for example the stomach, and after inflation, the internal retention assembly assumes an enlarged shape which prevents inadvertent removal of the gastric tube from the patient thereby allowing unimpeded fluid flow communication between the source of nutrition and the patient.

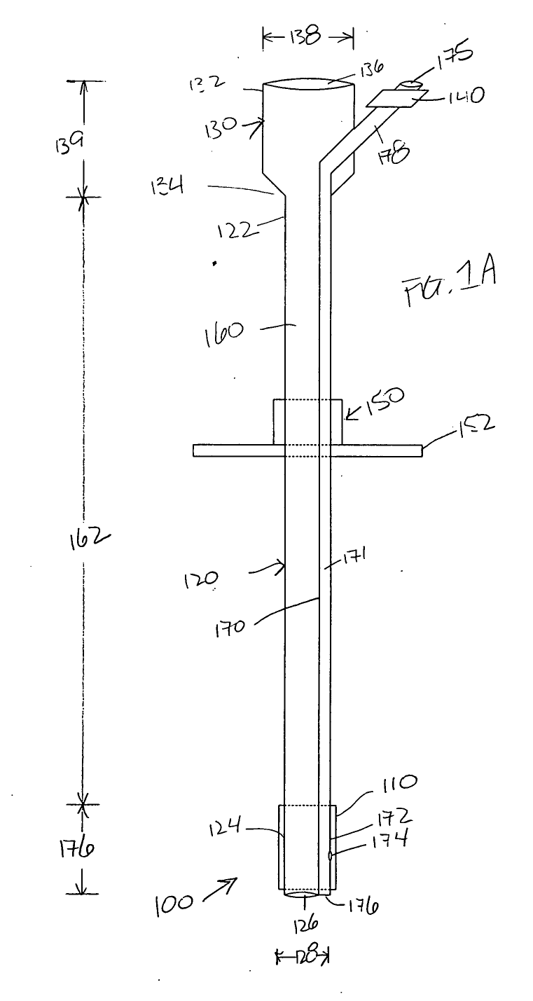

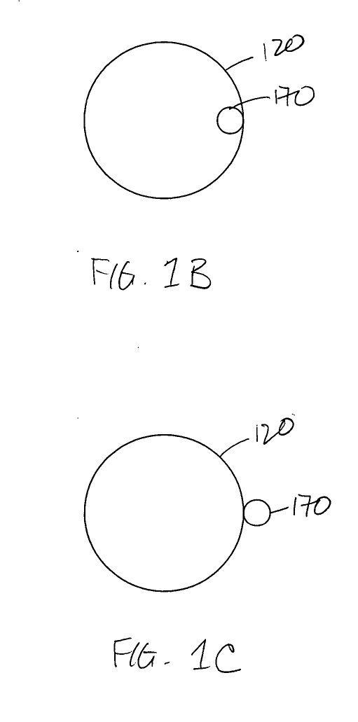

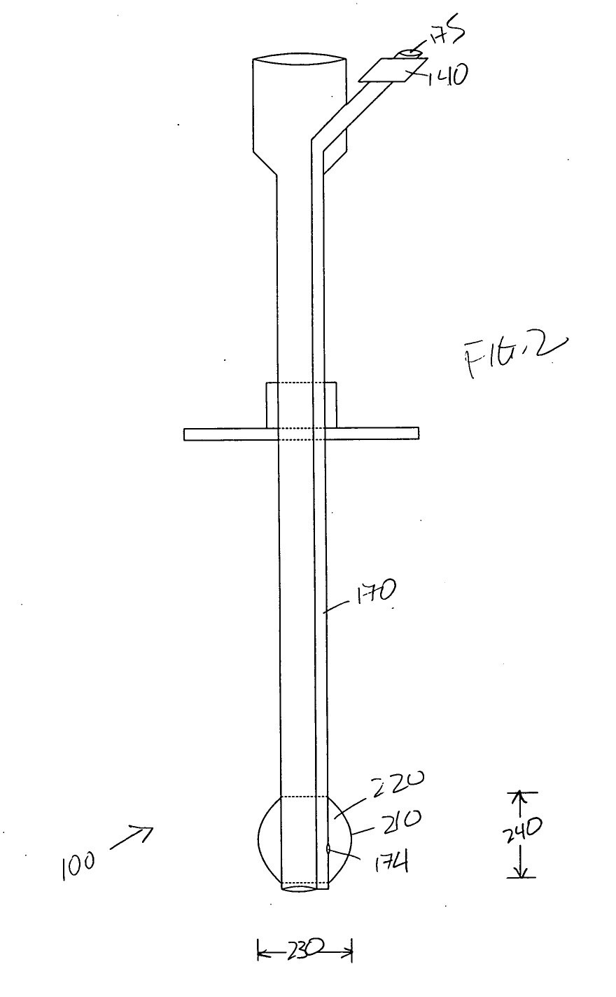

Referring now to FIG. 1A, Applicants' gastric tube 100 includes internal retention assembly 110, tubular member 120 comprising diameter 128, access assembly 130, and external retention assembly 150. Tubular member 120 includes proximal portion 122 and distal portion 124. Distal portion 124 is formed to include aperture 126.

Access assembly 130 includes proximal portion 132 and distal portion 134. Distal portion 134 comprises diameter 128 and is continuously attached to proximal portion 122 of first tubular member 120. Proximal portion 132 has diameter 138 and is formed to include aperture 136. First tubular member 120 in combination with access assembly 130 defines first lumen 160 which interconnects aperture 136 and aperture 126. When device 100 is inserted into a patient's stomach, fluids may be introduced into the stomach, or alternatively removed from the stomach, via lumen 160, aperture 126, and aperture 136.

Wound barrier 150 comprises flange 152. Wound barrier 150 is moveably disposed around tubular member 120 such that wound barrier 150 can be slidingly moved along member 120. In certain embodiments, wound barrier 150 is formed from a polyurethane. In certain embodiments, external retention assembly 150 is formed from natural rubber. In certain embodiments, wound barrier 150 is formed from a plasticized vinyl polymer. In certain embodiments, wound barrier 150 is formed from a non-fluorinated siloxane.

Tubular member 120 has a length 162. In certain embodiments, length 162 is less than one inch, for example in certain “low profile” NG tubes embodiments, to about 55 inches in certain J tube embodiments. In certain embodiments, length 162 is between about 6 inches and about 9 inches. In certain embodiments, length 162 is about 7 inches. Access portion 130 has a length 134. In certain embodiments, length 134 is between about 1.5 inches and about 3 inches. In certain embodiments, length 134 is about 2.25 inches.

In certain embodiments, tubular member 120 is formed from a polyurethane. In certain embodiments, tubular member 120 is formed from natural rubber. In certain embodiments, tubular member 120 is formed from a plasticized vinyl polymer. In certain embodiments, tubular member 120 is formed from a non-fluorinated siloxane.

Device 100 further includes tubular member 170 disposed within lumen 160. In certain embodiments, tubular member 170 is formed from a polyurethane. In certain embodiments, tubular member 170 is formed from natural rubber. In certain embodiments, tubular member 170 is formed from a plasticized vinyl polymer. In certain embodiments, tubular member 170 is formed from a non-fluorinated siloxane.

Tubular member 170 includes a proximal portion 178 comprising one-way valve 140, and formed to include aperture 175, where one-way valve 140 is disposed adjacent aperture 175. Proximal portion 178 extends through, and outwardly from, access assembly 130.

Tubular member 170 further includes distal portion 172 comprising closed end 176, where distal portion 172 is formed to include aperture 174 adjacent closed end 176. Closed end 176 is disposed adjacent aperture 126. In certain embodiments, closed end 176 includes a radio-opaque material, such as a barium-containing material. Tubular member 170 encloses second lumen 171 which interconnects aperture 174 and aperture 175.

Referring now to FIGS. 1B and 1C, in certain embodiments, tubular member 170 is disposed within tubular member 120 as shown in FIG. 1B. In certain embodiments, tubular member 170 is disposed external to tubular member 120 as shown in FIG. 1C. In certain embodiments, Applicants' device includes one or more tubular members disposed within tubular member 120 and/or one or more tubular members disposed external to tubular member 120.

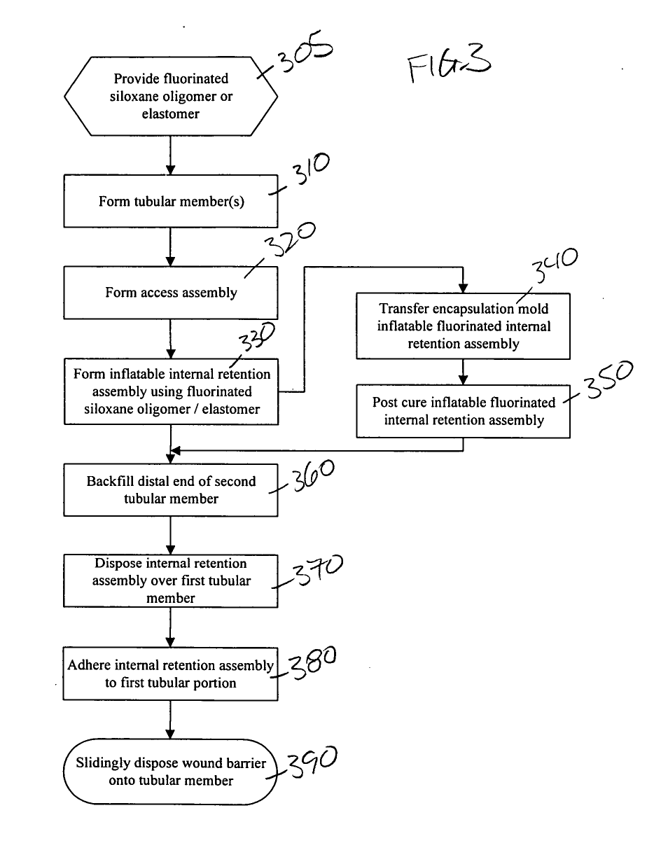

Referring now to FIG. 2, introduction of one or more inflating fluids 220 through aperture 175, through one-way valve 140, through tubular member 170, through aperture 174, and into internal retention assembly 110, causes assembly 110 to increase in dimension, i.e. to inflate, to form inflated assembly 210. One-way valve 140 permits, for example, one or more inflating fluids to be injected into aperture 175 and to flow outwardly from aperture 174, but does not permit those fluids to flow in the reverse direction, i.e. outwardly from aperture 175.

Such inflating fluids include, for example, one or more gases, such as air; one or more liquids, such as water, saline; and the like. The diameter of inflated assembly 210 is determined by the volume of fluid 220 introduced through aperture 175. Inflated internal retention assembly 210 has a diameter 230, and a length 240. In certain embodiments, diameter 230 is between about 1 inch and about 3 inches. In certain embodiments, length 240 is between about 1 inch and about 6 inches. In certain embodiments, the walls of inflated retention assembly 210 have a thickness of between about 0.005 inches and about 0.2 inches.

As those skilled in the art will appreciate, when in actual use the distal portion of Applicants' gastric tube, including inflated internal retention assembly 210, remains continuously disposed within a patient's stomach. Thus, inflated internal retention assembly 210 is continuously exposed to gastric fluid. Such gastric fluid contains pepsin, hydrochloric acid, mucin, and small quantities of various inorganic salts. This gastric fluid is extremely acidic, typically having a pH between about 0.9 and about 1.5.

As those skilled in the art will appreciate, it is desirable to leave a gastric tube in place for as long a time as possible to minimize patient distress upon removing one tube and inserting a replacement tube. As a practical matter, the useful lifetime of such a gastric tube is determined by the mechanical strength of the inflated internal retention assembly 210. Such an inflated internal retention assembly is continuously exposed to both mechanical stress and chemical stress.

Prior art inflatable internal retention assemblies typically comprise an elastomeric siloxane material, such as polydimethylsiloxane I.

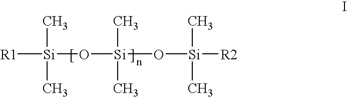

In certain embodiments, such prior art internal retention assemblies comprise a crosslinked elastomer formed from an oligomeric dimethylsiloxane I. By an “oligomeric” siloxane or an “oligomeric” fluorinated siloxane, Applicants mean a siloxane/fluorinated siloxane having a fixed volume but not a fixed shape, i.e. a liquid, at room temperature. By “fluorinated siloxane,” Applicants mean a material comprising alternating oxygen atoms and silicon atoms, i.e. a siloxane, where one or more of those silicon atoms is covalently bonded to a pendent alkyl chain comprising a plurality of carbon atoms, wherein at least one of those pendent carbon atoms is covalently bonded to one or more fluorine atoms.

Applicants have found that inflated internal retention assembly 210 formed from a fluorinated siloxane has increased resistance to acid, thereby giving Applicants' device an increased useful lifetime. In certain embodiments, Applicants' internal retention assembly 110/210 comprises an elastomeric trifluoropropylmethylsiloxane II, wherein R1 is selected from the group consisting of an alkyl group, an alkoxy group, an aromatic group, a vinyl group, an alkenyl group, an oxyalkenyl group [—O—(CH₂)ₙ—CH═CH₂]), and wherein R2 is selected from the group consisting of an alkyl group, an alkoxy group, an aromatic group, a vinyl group, an alkenyl group, and an oxyalkenyl group. In certain embodiments, R1 and R2 are the same. In certain embodiments, R1 and R2 differ. By “elastomer” or “elastomeric material,” Applicants mean a material which has a one hundred percent (100%) or greater elongation at room temperature using ASTM Test D412.

In certain embodiments, Applicants' internal retention assembly 110 comprises a crosslinked fluorinated elastomer formed from oligomeric trifluoropropylmethylsiloxane II.

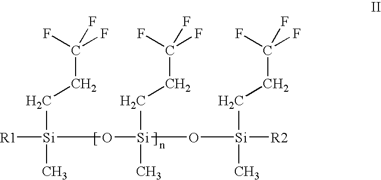

In embodiments wherein Applicants' internal retention assembly 110 is formed by molding an elastomeric trifluoropropylmethylsiloxane II, or is formed by crosslinking and molding an oligomeric trifluoropropyl methyl siloxane II, Applicants' internal retention assembly comprises a fluorinated siloxane which includes trifluoropropyl-methyl-siloxane repeat units IIA.

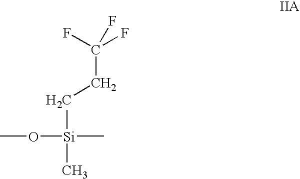

In certain embodiments, Applicants' gastric tube includes an inflatable internal retention assembly comprising a mixture of one or more dialkyl siloxanes, such as polydimethylsiloxane I, in combination with one or more fluorinated siloxanes, such as for example elastomeric siloxane II. In certain embodiments, Applicants' gastric tube includes an inflatable internal retention assembly formed using a mixture of an oligomeric dialkyl siloxane, such as oligomeric siloxane I, in combination with a fluorinated oligomeric siloxane, such as oligomer II, wherein R1 and R2 are as described above, and wherein (n) is less than or equal to 10. In certain embodiments, the weight percentage of the dialkyl siloxane oligomer/elastomer is between about 0 weight percent and about 99 weight percent, and the weight percentage of the fluorinated siloxane oligomer/elastomer is between about 1 weight percent and about 100 weight percent. Table I summarizes formulations A through S comprising the recited weight percentages of elastomeric siloxane I and elastomeric siloxane II.

TABLE I

| | A | B | C | D | E | F | G | H | I | J | K | L | M | N | O | P | Q | R | S |
|---|---|---|---|---|---|---|---|---|---|---|---|---|---|---|---|---|---|---|---|
| I | 99 | 90 | 85 | 80 | 75 | 70 | 65 | 60 | 55 | 50 | 45 | 40 | 35 | 30 | 25 | 20 | 15 | 10 | 0 |
| II | 1 | 10 | 15 | 20 | 25 | 30 | 35 | 40 | 45 | 50 | 55 | 60 | 65 | 70 | 75 | 80 | 85 | 90 | 100 |

In certain embodiments, Applicants' gastric tube includes an inflatable internal retention assembly comprising a copolymer which includes dialkyl siloxane repeat units and fluorinated siloxane repeat units. In certain embodiments, Applicants' gastric tube comprises an inflatable internal retention assembly comprising siloxane copolymer III.

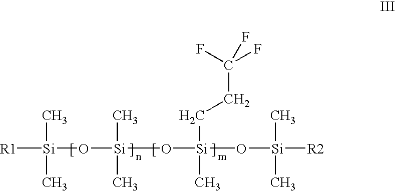

In certain embodiments, copolymer III comprises a random copolymer, wherein one or more trifluoropropylmethylsiloxane repeat units IIA are randomly distributed within copolymer III. By “randomly distributed within copolymer III,” Applicants mean that copolymer III includes a plurality of repeat units IIA dispersed throughout, where each repeat unit IIA is disposed between two repeat units IA.

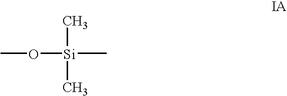

In certain embodiments, copolymer III comprises a block copolymer. In certain embodiments, (m) is about 10. In certain embodiments, (m) is greater than or equal to about 10. In certain embodiments, (m) is less than or equal to about 10. In certain embodiments, (n) is about 10. In certain embodiments, (n) is greater than or equal to about 10. In certain embodiments, (n) is less than or equal to about 10.

In certain embodiments, Applicants' device includes an inflatable internal retention assembly 110 formed from a fluorinated siloxane oligomer sold by Dow Corning under the tradename LS5-8725. In certain embodiments, internal retention assembly 110 is formed from LS5-8725, wherein LS5-8725 comprises oligomeric siloxane II, wherein R1 and R2 are vinyl, i.e. —CH═CH₂, and wherein (n) is 3. In these embodiments, internal retention assembly 110 consists essentially of a fluorinated siloxane elastomer comprising about 37 weight percent fluorine atoms. As those skilled in the art will appreciate, in these embodiments Applicants' internal retention assembly 110 may include small amounts of antioxidants, molding aids, and the like.

FIG. 3 summarizes the steps used to form Applicants' device. In step 305, Applicants' method provides a fluorinated siloxane oligomer or elastomer. In certain embodiments, step 305 includes providing an oligomer having structure II. In certain embodiments, step 305 includes providing an oligomer having structure III. In certain embodiments, step 305 includes providing an elastomer having structure II. In certain embodiments, step 305 includes providing an elastomer having structure III.

In step 310, Applicants' method forms tubular member 120. In certain embodiments, step 310 includes molding tubular member 120 from a non-fluorinated siloxane oligomer/elastomer. In certain embodiments, step 310 includes forming a tubular member comprising at least two lumens, such as first lumen 160 and second lumen 171.

In step 320, Applicants' method forms access assembly 130. In certain embodiments, step 320 includes molding access assembly 130. In certain embodiments, access assembly 130 is over molded onto tubular member 120.

In step 330, Applicants' method forms the inflatable, fluorinated internal retention assembly 110. In certain embodiments, step 330 includes molding a fluorinated elastomer to form internal retention assembly 110. In certain embodiments, step 330 includes encapsulation molding inflatable internal retention assembly 110. In certain embodiments, step 330 includes using other molding processes.

In certain embodiments, step 330 includes forming a molding composition comprising a fluorinated siloxane oligomer, such as oligomer II or oligomer III, in combination with a curing agent. The curing agent is present in an amount between about 0 weight percent and about 1.5 weight percent.

In certain embodiments, the curing agent comprises one or more transition metals. In certain embodiments, the curing agent comprises Platinum. In certain embodiments, the curing agent comprises one or more peroxides. In certain embodiments, the curing agent comprises Bis-(2,4-Dichlorobenzoyl) peroxide.

In these molding composition embodiments, step 330 further includes introducing Applicants' molding composition into a heated mold. The mold is heated to a temperature between about 125° F. and about 375° F. In certain embodiments, the clamping pressure used for the heated mold is about 5,000 psi. In certain embodiments, the clamping pressure used for the heated mold is about 40,000 psi. A transfer pressure between about 0 psi and about 10,000 is used. A molding time of up to about 6 minutes is used.

The heated mold activates the curing agent. The activated curing agent then causes chain extension and crosslinking of the fluorinated siloxane oligomer I/III to form a crosslinked, fluorinated siloxane elastomer.

Referring now to Reaction Sequence I below, in embodiments wherein the curing agent comprises a peroxide, and wherein R1 and R2 moieties of fluorinated siloxane oligomer II and/or fluorinated siloxane oligomer RIII comprise alkyl groups, such as methyl groups, peroxide V decomposes in reaction (1) to form 2 alkoxy radicals VI. In reaction (2), an alkoxy radical VI abstracts a proton from a methyl group on siloxane oligomer II/III to form a high energy fluorinated siloxane radical VII. In reaction (3) two of such high energy fluorinated siloxane radicals VII couple, i.e. chain extend/crosslink, to form a higher molecular weight compound VIII. As those skilled in the art will appreciate, Reaction Sequence I is repeated a plurality of times to form a polymeric, crosslinked, fluorinated elastomer.

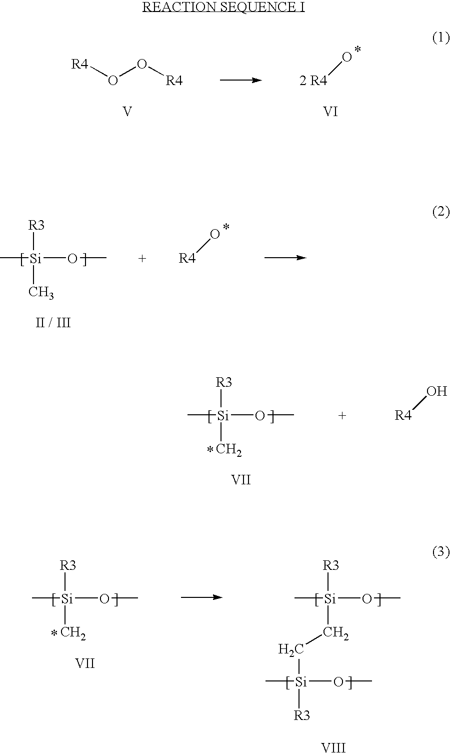

As those skilled in the art will appreciate, R3 is selected from the group consisting of an alkyl group, an alkoxy group, an aromatic group, a vinyl group, an alkenyl group, an oxyalkenyl group [—O—(CH₂)ₙ—CH═CH₂]). As those skilled in the art will further appreciate, R4 is selected from the group consisting of an alkyl group, an aromatic group, and combinations thereof. In certain embodiments, R1, R2, and R3 are the same. In certain embodiments, R1, R2, and R3 differ.

Referring now to Reaction Sequences I and II, in embodiments wherein the curing agent comprises a peroxide, and wherein R1 and/or R2 and/or R3 components of fluorinated siloxane oligomer II/fluorinated siloxane oligomer III comprises one or more vinyl groups, the peroxide curing agent V decomposes in reaction (1) to form 2 alkoxy radicals VI. In reaction (4), alkoxy radical VI adds to a pendent vinyl group on the fluorinated siloxane oligomer IX to form a high energy fluorinated siloxane radical X. In reaction (5), that high energy fluorinated siloxane radical X reacts with another fluorinated siloxane oligomer II/III to form a higher molecular weight/crosslinked fluorinated siloxane XI and an alcohol. As those skilled in the art will appreciate, Reaction Sequence II is repeated a plurality of times to form a polymeric, crosslinked, fluorinated elastomer.

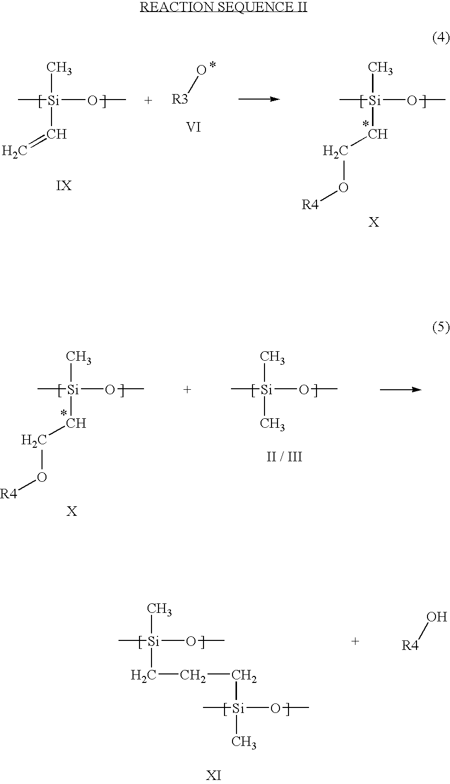

Referring again to FIG. 3, step 330 further includes removing the molded internal retention assembly from the heated mold. In certain embodiments, Applicants' method includes step 350 wherein the molded internal retention assembly is post cured after removal from the mold. Such a post cure can be performed immediately after molding, or up to several months after molding. Any heat generating source may be used for such post cure. In certain embodiments, the molded internal retention assembly is post cured for up to about 500 minutes at a post cure temperature between about 100° F. and about 600° F.

In step 360 Applicants' method backfills the distal end, such as distal end 176, of tubular member 170 disposed within tubular member 120. In certain embodiments, distal end 176 is backfilled with a radio-opaque material. In certain embodiments, the radio-opaque material comprises barium.

In step 370, Applicants' method disposes the internal retention assembly formed in step 330 around the first tubular member formed in step 310. In certain embodiments, step 350 includes advancing the distal end of tubular member through the center portion of a toroidal-shaped fluorinated internal retention assembly. In step 380, Applicants' method adheres the uninflated, fluorinated internal retention assembly 110 to first tubular member 120. In step 390, Applicants' method disposes the slideable wound barrier, such as external retention assembly 150, around tubular member 120.

The following Example is presented to further illustrate to persons skilled in the art how to make and use the invention and to identify presently preferred embodiments thereof. This Example is not intended, however, as a limitation upon the scope of Applicants' invention which is defined by the claims appended hereto.

### EXAMPLE

A prior art gastric tube was prepared using the method of FIG. 3 wherein the inflatable internal retention assembly 110 was formed using a vinyl-terminated dimethyl siloxane oligomer, such as oligomeric siloxane I wherein R1 and/or R2 are vinyl. This dimethyl siloxane based internal retention assembly was formed using a peroxide cure as described above.

Applicants' gastric tube 100 was prepared using the method of FIG. 3 wherein the inflatable internal retention assembly 110 was formed using oligomeric fluorinated siloxane II comprising about 37 weight percent fluorine atoms, wherein R1 and/or R2 are vinyl, and wherein R3 is methyl. Applicants' fluorinated siloxane internal retention assembly was formed using a peroxide cure as described above.

The inflatable internal retention assembly portions, i.e. the “balloon portions,” of both prior art gastric tubes and Applicants' gastric tubes were filled, i.e. inflated, with either 20 ml or 24 ml of water. Those inflated balloon portions of the prior art tubes and Applicants' tubes were then immersed in a simulated gastric fluid medium having a pH of about 1.2. This simulated gastric fluid was prepared using a USP formulation comprising 2.0 grams of NaCl, 3.2 grams of Pepsin, and 7.0 mL of 10% HCl solution. Water was added to bring the total volume to 1000 mL.

The day on which the first prior art inflated balloon burst was noted. In addition, the mean number of days for failure of all the prior art inflated balloons was determined. Table II summarizes the immersion test data for the prior art gastric tubes

TABLE II

| Inflation Volume, ml | Day, First Burst | Mean Days 100% Failure |
|---|---|---|
| 20 | 1 | 2 |
| 24 | 1 | 1 |

Similarly, the day on which the first fluorinated inflated balloon burst was noted. In addition, the mean number of days for failure of all the inflated fluorinated balloons was determined. Table III summarizes the immersion test data for Applicants' gastric tube.

TABLE III

| Inflation Volume, ml | Day, First Burst | Mean Days 100% Failure |
|---|---|---|
| 20 | 67 | >90 |
| 24 | 56 | >60 |

As the data of Tables II and III clearly shows. Applicants' internal retention assembly 110 comprising fluorinated siloxane elastomer II, when inflated, has greatly enhanced acid resistance compared to the prior art devices. Such increased acid resistance results in an increased useful lifetime for Applicants' device compared to prior art gastric tubes. Such increased an increased useful lifetime for Applicants' device, whether of the gastric type or of the nasogastric type or of the jejunal type, results in decreased distress and discomfort for patients using such a device.

While the preferred embodiments of the present invention have been illustrated in detail, it should be apparent that modifications and adaptations to those embodiments may occur to one skilled in the art without departing from the scope of the present invention as set forth in the following claims.

I claim:

1. A device for insertion into a hollow organ of an animal, comprising:
a first tubular member comprising a first diameter, a proximal portion, and a distal portion formed to include a first aperture;
a tubular access assembly comprising a proximal portion having a second diameter and formed to include a second aperture, and a distal portion having said first diameter, wherein said distal portion of said access assembly is continuously attached to said proximal portion of said first tubular member, and wherein said second diameter is greater than or equal to said first diameter;
an inflatable internal retention assembly disposed around said distal portion of said first tubular member, wherein said inflatable retention assembly comprises a fluorinated siloxane elastomer;
wherein said tubular access assembly in combination with said first tubular member defines a first lumen interconnecting said first aperture and said second aperture.

2. The device of claim 1, further comprising a wound barrier moveably disposed around said tubular member.

3. The device of claim 1, wherein said fluorinated siloxane elastomer comprises about 37 weight percent fluorine atoms.

4. The device of claim 3, wherein said fluorinated siloxane elastomer is formed from an oligomer comprising the structure:

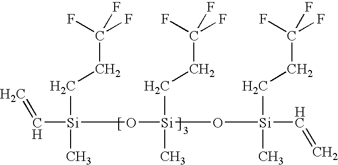

5. The device of claim 1, wherein said inflatable internal retention assembly comprises a mixture of a dialkylsiloxane elastomer and a fluorinated siloxane elastomer.

6. The device of claim 1, wherein said fluorinated siloxane comprises polytrifluoropropylmethylsiloxane.

7. The device of claim 1, wherein said fluorinated siloxane elastomer comprises a copolymer comprising trifluoropropylmethylsiloxane repeat units and dimethylsiloxane repeat units.

8. The device of claim 7, wherein said trifluoropropylmethylsiloxane repeat units are randomly dispersed throughout said copolymer.

9. The device of claim 7, wherein said copolymer comprises one or more blocks of polytrifluoropropylmethylsiloxane in combination with one or more blocks of polydimethylsiloxane.

10. The device of claim 1, further comprising:
a second tubular member disposed within said first lumen, wherein said second tubular member comprises a proximal portion comprising an open end and a one-way valve disposed adjacent said open end, and a distal portion comprising a closed end formed to include an aperture therethrough adjacent said closed end;
wherein said proximal portion of said second tubular extends through and outwardly from said access assembly, and wherein said closed end of said second tubular member is disposed adjacent said second open end of said first tubular member.

11. A method to form a device for insertion into a hollow organ of an animal, comprising the steps of:
providing a fluorinated siloxane;
molding a tubular member comprising a first diameter, a proximal portion, and a distal portion formed to include a first aperture;
molding a tubular access assembly comprising a proximal portion having a second diameter and formed to include a second aperture, and a distal portion having said first diameter, wherein said second diameter is greater than or equal to said first diameter;
continuously attaching said distal portion of said access assembly to said proximal portion of said first tubular member;
forming an internal retention assembly comprising said fluorinated siloxane;
adhering said inflatable internal retention assembly to said distal portion of said tubular member;
slidingly disposing a wound barrier onto said tubular member.

12. The method of claim 12, further comprising the step of post-curing said internal retention assembly.

13. The method of claim 11, wherein said providing a fluorinated siloxane further comprises providing a fluorinated siloxane comprising about 37 weight percent fluorine atoms.

14. The method of claim 13, wherein said forming an internal retention assembly step further comprises forming said internal retention assembly from an oligomer comprising the structure:

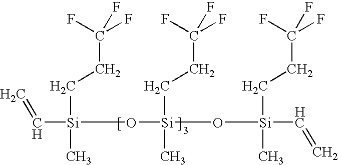

15. The method of claim 11, further comprising:
providing a dialkylsiloxane;
wherein said forming an internal retention assembly step further comprises forming said internal retention assembly from a mixture of said dialkyllsiloxane and said fluorinated siloxane.

16. The method of claim 11, wherein said providing a fluorinated siloxane step further comprises providing polytrifluoropropylmethylsiloxane.

17. The method of claim 11, wherein said providing a fluorinated siloxane step further comprises providing a copolymer comprising trifluoropropylmethylsiloxane repeat units and dimethylsiloxane repeat units.

18. The method of claim 17, wherein providing step further comprises providing a copolymer comprising trifluoropropylmethylsiloxane repeat units and dimethylsiloxane repeat units, wherein said trifluoropropyl-methyl-siloxane repeat units are randomly dispersed throughout said copolymer.

19. The method of claim 17, wherein providing step further comprises providing a copolymer comprising trifluoropropylmethylsiloxane repeat units and dimethylsiloxane repeat units, wherein said copolymer comprises one or more blocks of polytrifluoropropylmethylsiloxane in combination with one or more blocks of polydimethylsiloxane.
